(19) United States
(12) Patent Application Publication
(10) Pub. No.: US 2025/0000782 A1
(43) Pub. Date: Jan. 2, 2025

(54) XANTHONE-APPLIED TOOTHPASTE WITH BAMBOO SALT: PRESERVING THE PHARMACEUTICAL PROPERTY OF MANGOSTEEN PEEL

(71) Applicants: Meesue Kim, Kirkland, WA (US); Yoo Seung Kim, Bangkok (TH); Nam Soo Kim, Incheon (KR); Min Kyu Kim, Incheon (KR)

(72) Inventors: Meesue Kim, Kirkland, WA (US); Yoo Seung Kim, Bangkok (TH); Nam Soo Kim, Incheon (KR); Min Kyu Kim, Incheon (KR)

(73) Assignee: Meesue Kim, Woodinville, WA (US)

(21) Appl. No.: 18/343,691

(22) Filed: Jun. 28, 2023

(51) Int. Cl. A61K 8/9789; A61K 8/49; A61K 8/9794; A61Q 11/02

(52) U.S. Cl. CPC A61K 8/9789; A61K 8/498; A61K 8/9794; A61Q 11/02; A61K 2800/522; A61K 2800/70; A61K 2800/87; A61K 2800/92

(57) ABSTRACT

This invention outlines a method for creating a xanthone-enriched toothpaste using mangosteen peel and bamboo salt. This involves obtaining and preserving mangosteen peel, extracting xanthones from the peel, and incorporation with bamboo salt into a toothpaste formula. The product is designed for efficient absorption into gum cells during brushing, leveraging xanthones and bamboo salt to penetrate dental biofilm and absorption through oral mucosa, and provides antibacterial, anti-inflammatory, and antioxidant effects. Xanthones are proven to inhibit tooth decay and gum disease causing oral bacteria like Streptococcus mutans and Porphyromonas gingivalis. Mineral-rich bamboo salt adds therapeutic value to the toothpaste. The unique combination in the toothpaste helps target and combat oral health issues by reducing pro-inflammatory cytokines production, inhibiting harmful bacterial activity, and enhancing oral tissue health, with direct delivery to the mouth during brushing. This invention promotes oral health naturally by fighting harmful bacteria, reducing inflammation, and offering mineral enrichment.

### FIELDS

The present invention relates to the fields of oral care, biomass treatment, manufacturing of oral care products, processing of herbs, and chemical extraction. More specifically, the invention pertains to a method for producing xanthone-applied toothpaste using mangosteen peel as a source of xanthones, combined with the incorporation of bamboo salt. The invention encompasses the preservation and extraction of xanthones from mangosteen peel, the formulation of a toothpaste incorporating the extracted xanthones and bamboo salt, and the utilization of the toothpaste to promote oral health. By combining expertise from various fields, including oral care, biomass treatment, manufacturing, processing of herbs, and chemical extraction, the invention provides an innovative approach to enhance the effectiveness of toothpaste in preventing oral diseases, reducing inflammation, and promoting overall oral health.

### BACKGROUND OF THE INVENTION

The present invention relates to the field of oral care and, more specifically, to a method for producing xanthone-applied toothpaste using mangosteen peel as a source of xanthones, along with the incorporation of bamboo salt as an additional ingredient. Maintaining good oral health is essential for overall well-being. Proper oral hygiene practices, including regular brushing and the use of toothpaste, are crucial in preventing oral diseases such as tooth decay and gum disease. Traditional toothpastes typically contain a combination of abrasives, surfactants, and flavoring agents, with limited emphasis on utilizing natural ingredients that offer additional therapeutic benefits. Mangosteen (Garcinia mangostana) is a tropical fruit known for its rich content of xanthones, a class of natural compounds with various biological activities. Xanthones have been widely studied for their antimicrobial, anti-inflammatory, and antioxidant properties. These properties make xanthones a promising candidate for incorporation into oral care products, as they can potentially enhance their effectiveness in promoting oral health. The extraction of xanthones from mangosteen peel presents a unique opportunity to utilize this natural resource for oral care applications. Furthermore, the present invention incorporates bamboo salt, known for its rich mineral content and unique processing method, into the toothpaste formulation. Bamboo salt adds therapeutic benefits to the toothpaste, including minerals that nourish the oral tissues and contribute to overall oral health. Mangosteen peel, which is often discarded as waste, contains a significant amount of xanthones that can be extracted and incorporated into toothpaste formulations. By utilizing the xanthone-rich extract from mangosteen peel and the unique properties of bamboo salt, the toothpaste offers enhanced antibacterial, anti-inflammatory, and antioxidant effects, providing additional benefits beyond conventional toothpastes. The present invention aims to overcome the limitations of traditional toothpastes by introducing a method for producing xanthone-applied toothpaste using mangosteen peel as a source of xanthones, combined with the therapeutic properties of bamboo salt. By capitalizing on the antimicrobial, anti-inflammatory, and antioxidant properties of xanthones and the beneficial minerals in bamboo salt, the present invention offers a natural and scientifically supported approach to improving oral health. In summary, the revised background of the invention highlights the need for improved oral care products that harness the therapeutic properties of natural ingredients, including xanthones from mangosteen peel and bamboo salt. The utilization of mangosteen peel and bamboo salt in toothpaste formulation represents a novel and innovative approach to promoting oral health. By incorporating these ingredients, the present invention provides a valuable contribution to the field of oral care by offering a toothpaste with enhanced antimicrobial, anti-inflammatory, and antioxidant effects, along with beneficial minerals for overall oral health.

### BRIEF SUMMARY OF THE INVENTION

The present invention is a method for producing xanthone-applied toothpaste using mangosteen peel and bamboo salt. It involves sorting raw materials, preserving mangosteen peel's quality, extracting xanthones, and formulating them into a toothpaste. The toothpaste is designed for easy absorption by gum cells, delivering xanthones' antimicrobial, anti-inflammatory, and antioxidant benefits. Bamboo salt adds essential minerals for oral health. This invention offers a natural and scientifically supported approach to enhance oral well-being.

### DETAILED DESCRIPTION OF THE INVENTION

The invention provides a method for producing bamboo salt and xanthone-applied toothpaste using mangosteen peel as a source of xanthones. The method includes a series of steps that are designed to preserve the quality and pharmaceutical property of the mangosteen peel, extract xanthones from the preserved peel, and formulate the extracted xanthones and combining with bamboo salt into a toothpaste that can be easily absorbed via the membrane cells in the epithelial structure of the gum during brushing of the teeth.

The method includes the following steps:
1. Sorting the raw materials including bamboo salt and mangosteen peel.
   a) Removing the flesh from the mangosteen peel to obtain the biomass of mangosteen peel.
   b) Subjecting the biomass of mangosteen peel to low frequency nano-ozone bubble water cleansing technology to remove impurities and microbials from the surface of the peel.
   c) Treating the biomass of mangosteen peel with cold plasma vortex magnetic field to maintain the original pharmaceutical property of the mangosteen peel longer and cleaner.
   d) Vacuum packaging the treated biomass of mangosteen peel to prevent deterioration during storage and transport.
2. Extracting xanthones from preprocessed mangosteen peel:
   a) Obtaining xanthone essence by CO₂ supercritical extraction from the raw materials which are preprocessed via nano-ozone bubble water and cold plasma vortex magnetic fields, with the resulting xanthone essence comprising less than 2% xanthone. Then apply the residues after obtaining the xanthone to combine it together with bamboo salt.
   b) Mix the residues after obtaining the xanthone extraction by CO₂ supercritical extraction together with “Celtic sea salt” then filling it into the empty space of the stem of 3-years old “Japanese Cane bamboo” and bake whole at the temperature of 2,800° F. for over 20 hours. Grind rock-solid baked bamboo salt and refilling it into the space of a new bamboo stem and bake it again under the same conditions applied previously. Keep repeating above mentioned baking procedures for 11 times and grind the final baked rock-solid bamboo salt into the find powder.
   c) Obtaining another group of Xanthone essence by and from ethanol solvent extraction and chromatography of preprocessed mangosteen peel, with the resulting Xanthone essence comprising less than 3%
3. Formulating bamboo-salt and xanthone-applied toothpaste:
   a) Combining the xanthone essence, 11 times baked bamboo salt powder and Nano-size mangosteen peel powder with conventional toothpaste ingredients, including abrasives and surfactants, that are suitable for use in a toothpaste formulation.
   b) Formulating the bamboo salt & xanthone-applied toothpaste to be absorbed easily via the membrane cells in the epithelial structure of the gum during brushing of the teeth, and to function to calm down inflammation of the gum structure.

1. A toothpaste formulation comprising preserved mangosteen pericarp obtained through ozone treatment and low-frequency ultrasound in a modified atmosphere packaging (MAP) system, xanthone essence extracted from the preserved mangosteen pericarp, bamboo salt obtained through multiple cycles of baking 3 years old Japanese Cane-bamboo stems filled with Celtic Sea salt powder, and conventional toothpaste ingredients selected from the group consisting of abrasives, surfactants, flavorings, and preservatives.

2. The toothpaste formulation according to claim 1, wherein the preserved mangosteen pericarp retains its bioactive properties, including antioxidant, anti-inflammatory, and antimicrobial activities.

3. The toothpaste formulation according to claim 1, wherein the xanthone essence comprises a concentration of less than 2% xanthone.

4. The toothpaste formulation according to claim 1, wherein the bamboo salt is obtained by repeatedly baking the 3 years old Japanese Cane bamboo stems filled with “Celtic Sea salt” powder together with the residues obtained after extracting the xanthone from mangosteen peel at a temperature of 2,800° F. for a total of 9 cycles.

5. The toothpaste formulation according to claim 1, wherein the preserved mangosteen pericarp, xanthone essence, and bamboo salt are present in a weight ratio of 40-60% preserved mangosteen pericarp, 30-50% xanthone essence, and 10-30% bamboo salt.

6. The toothpaste formulation according to claim 1, wherein the preserved mangosteen pericarp, xanthone essence, and bamboo salt are homogeneously mixed with the conventional toothpaste ingredients.

7. A method for producing the toothpaste formulation according to claim 1, comprising the sorting the raw materials including mangosteen pericarp, xanthone essence, and bamboo salt, preserving the mangosteen pericarp through ozone treatment and low-frequency ultrasound in a modified atmosphere packaging (MAP) system, extracting xanthone essence from the preserved mangosteen pericarp, and obtaining bamboo salt through multiple cycles of baking bamboo stems filled with bamboo salt powder, and the step of pre-processing the raw materials including mangosteen pericarp, xanthone essence, and bamboo salt prior to preservation, extraction, and baking.

8. The method according to claim 7 wherein the preserved mangosteen pericarp retains its bioactive properties, including antioxidant, anti-inflammatory, and antimicrobial activities.

9. The method according to claim 7, wherein the xanthone essence comprises a concentration of less than 2% xanthone.

10. The method according to claim 7 wherein the bamboo salt is obtained by repeatedly baking bamboo stems filled with bamboo salt powder at a temperature of 2,800° F. for a total of 9 cycles.

11. The method according to claim 7, wherein the preserved mangosteen pericarp, xanthone essence, and bamboo salt are homogeneously mixed with the conventional toothpaste ingredients.

12. Use of the toothpaste formulation according to claim 1 for improving oral health, wherein the preserved mangosteen pericarp, xanthone essence, and bamboo salt work together to reduce the number of oral bacteria, prevent tooth decay, promote gum health, and provide overall oral hygiene benefits.

13. A packaging material for preserving the toothpaste formulation according to claim 1, comprising an ozone barrier material to prevent ozone leakage from the package, a low-frequency ultrasound transmissible material to allow ultrasound treatment of the toothpaste formulation, an oxygen barrier material to maintain a modified atmosphere within the package, and a material that is compatible with food contact regulations and provides the required barrier properties.

14. The packaging material according to claim 13, wherein the packaging material is composed of plastics, films, laminates, or coatings.

15. The packaging material according to claim 13, wherein the packaging material is transparent, translucent, or opaque.

16. A method for producing the packaging material according to claim 13, comprising the steps of selecting materials suitable for the ozone barrier, low-frequency ultrasound transmissible, and oxygen barrier layers, preparing the materials in a suitable form for packaging, (c) assembling the layers to form the packaging material, and (d) customizing the packaging material in terms of thickness, size, shape, and design.
